(19) United States
(12) Patent Application Publication
(10) Pub. No.: US 2023/0138992 A1
(43) Pub. Date: May 4, 2023

(54) Magnetic Shape-Forming Surgical Continuum Manipulator

(71) Applicant: University of Leeds, Leeds Yorkshire (GB)

(72) Inventors: Pietro Valdastri, Leeds Yorkshire (GB); James Henry Chandler, Leeds Yorkshire (GB); Giovanni Pittiglio, Leeds Yorkshire (GB); Peter Robert Lloyd, Leeds Yorkshire (GB); Tomás Guerreiro Da Veiga, Leeds Yorkshire (GB); Zaneta Milena Koszowska, Leeds Yorkshire (GB); Onaizah Onaizah, Leeds Yorkshire (GB); Russell Anthony Harris, Leeds Yorkshire (GB); Heather Elizabeth Owston, Leeds Yorkshire (GB); Stephen John Russell, Leeds Yorkshire (GB)

(21) Appl. No.: 17/913,427

(22) PCT Filed: Mar. 24, 2021
(86) PCT No.: PCT/GB2021/050717

§ 371 Date: Sep. 21, 2022

(30) Foreign Application Priority Data
Mar. 24, 2020 (GB) 2004276.8
Oct. 23, 2020 (GB) 2016819.1

(51) Int. Cl. G01R 33/383; A61B 34/00

(52) U.S. Cl. CPC G01R 33/383; A61B 34/70

(57) ABSTRACT

Magnetic shape-forming surgical continuum manipulator (“CM”) comprising an elastomeric base material and a plurality of magnetic elements, the plurality of magnetic elements being located at a plurality of points along a length of the CM and each magnetic element having a predetermined magnetic profile, whereby the shape of the CM can be magnetically manipulated substantially along said length by the application of an external magnetic field and a magnetic field gradient.

This application relates to the field of magnetically actuated shape-forming surgical continuum manipulators, methods of manufacture and methods of operation thereof.

### BACKGROUND

Surgical continuum manipulators (“CMs”) have been used to assist with and enable surgical procedures in the form of catheters and endoscopes for at least the last 120 years. Traditional continuum manipulators rely on body rigidity to transmit forces and torques from proximal to distal ends. This approach relies on operator skill, offers limited accuracy or dexterity and the process itself can cause tissue trauma.

These limitations may be mitigated with the use of soft robotic manipulators which are primarily fabricated from elastomeric materials. Such robotic manipulators may be fluid driven, tendon driven, made from shape memory alloy or electroactive polymer, or magnetically actuated.

Tip driven magnetically actuated CMs wherein the tip of the device is magnetically driven have been demonstrated to increase control and reduce trauma during the negotiation of anatomical convolutions. Example are described in: S Jeon, AK Hoshiar, K Kim, S Lee, E Kim, S Lee, J-y Kim, BJ Nelson, H-J Cha, B-J Yi and H Choi, “A Magnetically Controlled Soft Microrobot Steering a Guidewire in a Three-Dimensional Phantom Vascular Network”, Soft Robotics, vol 6, no 1, pp54-68, October 2018 https://doi.org/10.1089/soro.2018.0019, and Y Kim, GA Parada, S Liu and X Zhao, “Ferromagnetic soft continuum robots”, Science Robotics, vol 4, no 33, p.eaax7239,2019.

These systems, however, can only assume the body shape of their respective conduit via anatomical interactions. The highly convoluted geometries and millimetre scale workspaces make this a challenging area of research and magnetic actuation has its own attendant complexities regarding the modelling and simulation of long, slender and potentially unstable elastomers.

It is therefore an object of the present invention to provide an improved magnetically actuated shape-forming surgical continuum manipulator.

### BRIEF SUMMARY OF THE DISCLOSURE

The invention is defined in the appended claims. According to a first aspect of the invention, there is provided a magnetic shape-forming surgical continuum manipulator (“CM”) comprising an elastomeric base material and a plurality of magnetic elements, the plurality of magnetic elements being located at a plurality of points along a length of the CM and each magnetic element having a predetermined magnetic profile, whereby the shape of the CM can be magnetically manipulated substantially along said length by the application of an external magnetic field and, optionally, a magnetic field gradient.

In an embodiment, the plurality of magnetic elements comprises magnetic particles dispersed in the elastomeric base material. The magnetic particles may be dispersed at different concentrations and/or have different magnetic profiles along said length.

In another embodiment, the plurality of magnetic elements comprises multiple spaced permanent magnets embedded in the elastomeric base material.

In an embodiment, the shape-forming surgical continuum manipulator further comprises a lumen along said length providing a working channel therethrough. Optical fibres for laser ablation, for example, could be provided and operated via said lumen.

Preferably, the magnetic shape-forming surgical continuum manipulator has an external diameter of less than 2 mm.

In an embodiment, the magnetic shape-forming surgical continuum manipulator further comprises one or more sensors.

In an embodiment, the elastomeric base material has an anisotropic elasticity distribution which can improve bending performance of the CM by reducing torsion.

The magnetic shape-forming surgical continuum manipulator may further comprise a reinforcing element having higher stiffness than said elastomeric based material. The reinforcing element may comprise a helical element.

According to a second aspect of the invention, there is provided a method of manufacturing a magnetic shape-forming surgical continuum manipulator according to any of the preceding paragraphs comprising the steps of:
a. Combining said magnetic elements with the elastomeric material by dispersing or embedding said magnetic elements therein; and
b. Magnetizing said magnetic elements to create said predetermined magnetic profile.

In an embodiment, the combining step comprises extruding said elastomeric material. Alternatively, the combining step comprises moulding said elastomeric material in a shaped tray.

The combining step may be performed before, after or during said magnetizing step.

According to a third aspect of the invention, there is provided a method of controlling a magnetic shape-forming surgical continuum manipulator according to any of the preceding paragraphs comprising the steps of:
a. applying an external magnetic field to the CM;
b. allowing the CM to adopt a shape along the length thereof as a result of manipulating said external magnetic field.

“Manipulating” said external magnetic field may simply mean switching the field on or off, and/or may mean applying a magnetic field gradient.

In an embodiment, the method further comprises the step of pulling the CM to a new location as a result of the application and/or manipulation of said external magnetic field. Preferably, a pulling force is applied along the length of the CM.

In an embodiment, in step b, the CM adopts a stiffened shape in order to provide a working channel via said lumen. Alternatively, in step b, the CM adopts a dynamically changing shape dependent on said manipulation of the external magnetic field.

In an embodiment, said external magnetic field is applied by dual arm collaborative magnetic manipulation, electromagnetic coils or magnetic resonance imaging (MRI).

### BRIEF DESCRIPTION OF THE DRAWINGS

Embodiments of the invention will now be more particularly described, by way of example only, with reference to the accompanying drawings in which:

FIG. 1 is a side view of a CM according to an embodiment of the invention;

FIG. 2 is a side view of a CM according to another embodiment of the invention;

FIGS. 3A- 3D and 4 illustrate manufacturing methods for prototype CMs according to an aspect of the invention;

FIG. 5 is a schematic representation of dual arm control of a CM; and

FIG. 6 illustrates an extrusion and magnetisation method for prototype CMs.

FIGS. 7A - 7D show a fabrication process for a CM section with a helical reinforcement element.

FIGS. 8A and 8B show a CM without a helical reinforcement element and a CM with a helical reinforcement element.

### DETAILED DESCRIPTION

Throughout the description and claims of this specification, the term “continuum manipulator” or “CM” is intended to refer to a surgical continuum manipulator, tentacle or robotic manipulator having an elongate shape which can be manipulated. The definition extends to prototypes of any of the above, including those prototypes which have no surgical function.

Throughout the description and claims of this specification, the term “shape forming” is intended to refer to the property of a CM whereby its shape, in particular its curvature, can be selected, controlled or manipulated along part or all of its length.

The “proximal” end of a CM means the tail end of the CM, the end nearest the point of origin and nearest the clinician.

The “distal” end of a CM means the leading end of the CM, the end furthest from the point of origin and furthest from the clinician.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

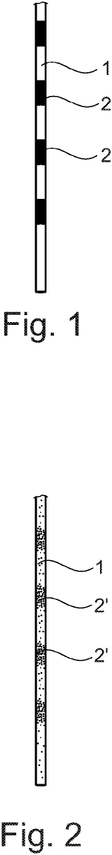

Referring to FIG. 1, a “multi-segment” magnetic shape-forming continuum manipulator (“CM”) is shown comprising an elastomeric base material 1 and a plurality of magnetic elements 2 embedded therein. The elastomeric base material 1 is a silicone elastomer such as Ecoflex™ 00-30. The magnetic elements 2 are equispaced permanent magnets made, for example, from silicone elastomer doped with neodymium-iron-boron (NDFeB) microparticles with an average diameter of 5 µm. The permanent magnets can each have their own individual magnetisation direction. Onboard sensors such as Hall effect sensors and IMUs (inertial measurement units) may be integrated or co-located with the permanent magnets so that they are spaced along the CM.

In an alternative embodiment shown in FIG. 2, a “single segment” CM comprises an elastomeric base material 1 doped throughout with a plurality of magnetic elements in the form of a plurality of magnetic particles. Optionally there may be regions 2′ having a greater concentration of magnetic particles. In manufacturing a prototype, particles of NdFeB were added to a prepolymer in a 1:1 ratio by weight equating to a volumetric ratio of 0.88:0.12 (Ecoflex™:NdFeB). The composite was mixed and degassed in a high vacuum mixer (ARV-310, THINKYMIXER, Japan) at 1400 rpm, 20.0 kPa for 90 seconds and then injected onto a straight cylindrical mould of diameter d=1.5 mm and length 20 mm and left to cure. The mould contained a centrally aligned 0.25 mm diameter Nitinol needle running for 10 mm of its length. This needle remained embedded in the polymer and was used to suspend and constrain the prototype during testing. Once the polymer had cured, the prototype was subjected to a uniform field of 46.44 KGauss (4.644 T) (ASC IM-10-30, ASC Scientific, USA) orthogonal to the CM prototype’s principle (longitudinal) axis.

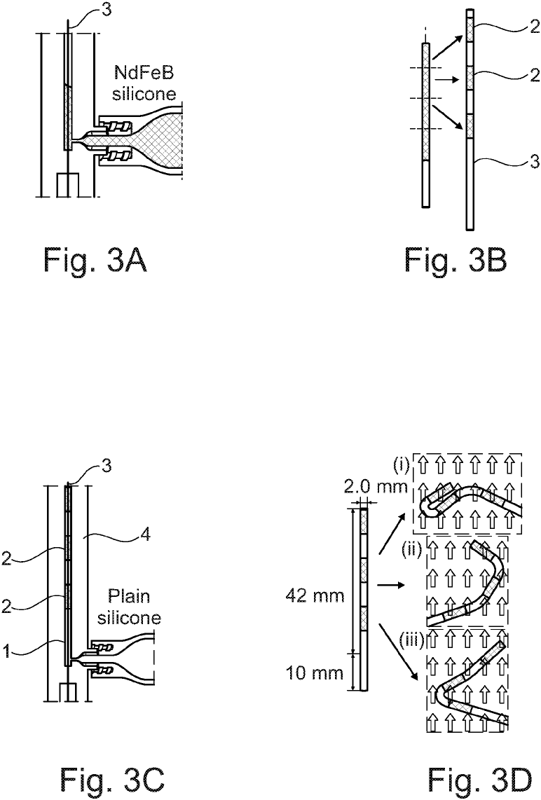

Referring to FIGS. 3A-3D, a prototype multi-segment CM was manufactured as follows. An unmagnetized elastomer doped with NdFeB was injected into a mould around a centrally aligned needle 3 (FIG. 3A). Once cured, the doped elastomer was divided into three identical 7 mm segments 2 which were axially separated by 14 mm, still on the needle (FIG. 3B). Alternatively the doped elastomer was removed from the needle 3 and divided into segments which were then replaced, axially spaced, on a needle of slightly greater diameter so that the axial positioning could be more easily maintained owing to the tighter friction fit.

The needle-mounted segments 2 were then placed in a second mould 4 and an undoped silicone elastomer base material 1 (Ecoflex™ 00-30) was injected around them (FIG. 3C). Upon curing of the polymer, the needle 3 was removed save for the final 10 mm which remained embedded to act as a mechanical constraint during experiments on the prototype.

The total length of the multi-segment prototype was 52 mm (FIG. 3D). From bottom to top this can be broken down as 10 mm of unconstrained length followed by 42 mm of constrained length. In the Figures, the undoped elastomer appears white and the doped segments comprising the magnetic elements 2 appear black. The dimensional accuracy of the fabricated CM prototypes was assessed through image analysis software (LAZ, EZZ, Leica, Germany), calibrated against a known reference length with images obtained using a digital light microscope (DMS300, Leica, Germany). The magnetic element segments 2 had lengths (Mean+/-SD) of 7.4+/-0.43 mm and diameters 1.9+/-0.03 mm. Specific values and dimensions mentioned above are given by way of example only and are not intended to limit the scope of the appended claims.

Instead of using a needle 3 to maintain the desired axial spacing of the segments 2, this could instead be achieved by the design of the mould shape per se and/or the use of radial pins or other alignment features to hold the segments in place as the elastomer base is moulded around them.

Using the above described method, a CM is manufactured by pre-preparing the magnetic elements 2 and then moulding the undoped elastomer 1 around the magnetic elements 2.

An alternative is to combine the elastomer with sequentially inserted magnetic elements as illustrated in FIG. 4. The doped elastomer magnetic segments 2 are prepared in the same way as described above in relation to FIGS. 3A and 3B and then removed from any supporting needle 3. A magnetic segment 2 is inserted into a mould 4 and then pushed down into the mould by the injection therein of undoped elastomer 1. Sequential alternate injection of elastomer and insertion of magnetic elements creates a CM with a desired distribution and spacing of magnetic elements 2 in an elastomeric base material 1. Once fully cured, the CM is removed from the mould 4.

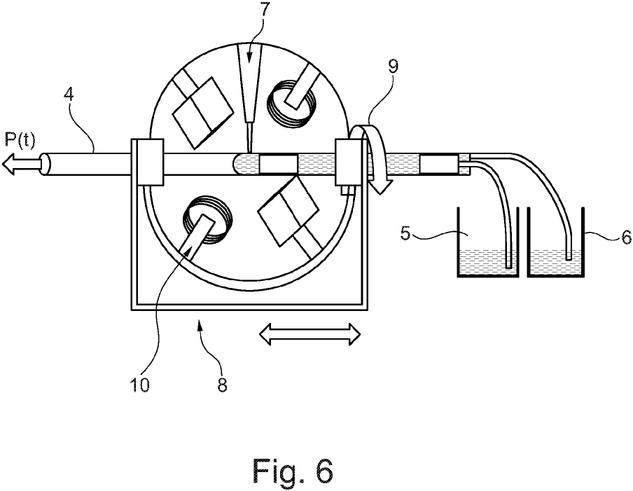

Instead of combining the undoped elastomer with the magnetic elements in one of the methods as described above, a further alternative is to extrude undoped elastomer simultaneously with doped elastomer as illustrated in FIG. 6. FIG. 6 shows a vacuum based extrusion system in which P(t) is applied to selectively extract liquid elastomer (either doped or undoped) from two reservoirs into a tube-shaped mould 4. Doped elastomer in liquid form is provided in reservoir 5. Undoped elastomer in liquid form is provided in reservoir 6. Doped or undoped elastomer can be drawn alternately into the mould 4, or a mixture can be simultaneously drawn from both reservoirs 5, 6, in order to create a desired concentration of doped particles along the length of the CM. Such a continuous distribution may be homogenous or may vary in concentration along the length of the CM.

The apparatus 8 provides localised curing of the CM for example using locally-applied heat or UV from curing apparatus 7. At least part of the apparatus 8 is rotatable about the longitudinal axis of the mould 4 (i.e. in the direction indicated by the arrow 9 in FIG. 6) so that heat/UV can be applied as desired.

The mould 4 may move through the apparatus 8, or the apparatus 8 may be linearly translated with respect to the mould 4.

The mould may comprise PVA so that it can easily be removed from the cured CM by dissolving the mould in water.

When manufacturing the “single segment” CM of FIG. 2, instead of injecting the composite into a mould as described above, an alternative method is to use a mould formed from a sacrificial gelatin. A cavity of desired shape is formed in a sacrificial gelatin and then the composite (the elastomer and magnetic particle mix) is injected into the sacrificial gelatin mould which supports the composite while it cures. Once cured, a magnetizing step (described below) is performed, after which the sacrificial gelatin mould can be removed by dissolving in hot water, leaving the single segment CM ready for use.

A magnetising step is employed to magnetise the magnetic elements of the CM prior to use in a clinical situation. The CM may be housed in a magnetizing tray (FIG. 3D) and exposed to for example a 46.44 KGauss (4.644 T) saturating field. The geometry of the magnetizing tray may be determined by the solution to the inverse static problem for the CM, the solution being generated by a neural network based on a predefined desired shape for the CM.

It is possible to perform the magnetising step of magnetising the doped segments/magnetic elements either before or after the moulding/extrusion step combining the elastomer and doped segments together. As illustrated in FIG. 6, it is also possible to perform the magnetising step simultaneously with extrusion using magnetising coil 10.

The elastomer may be moulded or extruded around a removable rod or needle which, when removed, leaves a lumen that can be used as a working channel.

The result is a CM having multiple magnetic elements arranged along its length i.e. not only at its distal tip as is conventionally known. Application of an external magnetic field and optionally a magnetic field gradient means the CM can be driven along a predetermined path by forces applied along its length so that it can be guided carefully through the desired path rather than pushed from the proximal end or pulled from the distal tip. The soft elastomer minimises trauma to surrounding tissues.

The CM may have a generally circular cross-sectional shape although other cross-sectional shapes are possible.

The diverse range of magnetic fields that will be applied to the CM could potentially lead to instability resulting from the CM twisting about its longitudinal axis in search for the minimum energy pose. Adaptive dynamic control of the applied magnetic fields could potentially be used to counteract this instability but this is impractical for real life applications due to the challenges of monitoring and sensing within the human body. An alternative solution is for the CM to have an anisotropic elasticity distribution by reinforcing the elastomer with higher stiffness fibres in order to restrict torsion whilst still permitting bending.

The CM may thus be provided with a helical reinforcing element. The helical reinforcing element 20 may be in the form of a single helix or a double helix (i.e. a pair of helices comprising one left handed helix and one right handed helix).

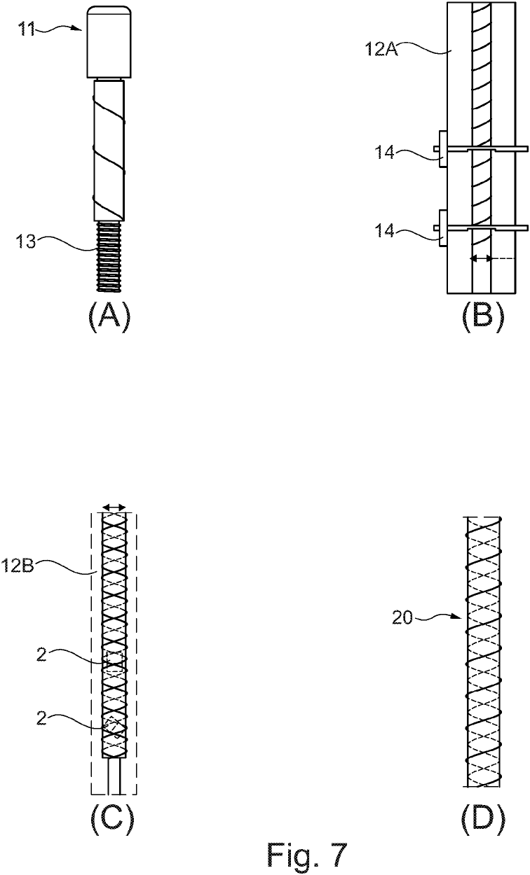

Steps for forming a CM with helical reinforcing element 20 are shown in FIG. 7. As shown in FIG. 7A, the helical reinforcing element 20 is made from extruded PLA (polylactide) fibre of diameter 0.4 mm (+ or - 0.02 mm) wound around a 3D printed cylindrical form 11 featuring the desired helical groove. The fibre is wound around the form 11 and secured before being subjected to a heat cycle peaking at 60° C. for 30 minutes. The heat treatment enables the fibre to retain the desired helical shape after removal from the form. This can be repeated for both left and right-handed helices with a helix angle of θ_{H} = 85°.

As shown in FIG. 7B, a clockwise helix is secured in a first cylindrical mould 12A., Cylindrical inserts 14 are provided at intervals to create cavities at predefined desired angles for the magnetic elements 2 to be inserted later. The elastomeric base material is injected into the mould around the clockwise helix and cured.

One removed from the first mould 12A, the cured structure is placed within a second, anti-clockwise helix and the inserts 14 are removed so that magnetic elements 2 (permanent magnets) can be placed in the resulting cavities. Next, as shown in FIG. 7C, the structure is placed in a second mould 12B so that additional elastomeric base material can be injected to secure the magnetic elements and anti-clockwise helix in place. The completed reinforced CM 20 is shown in FIG. 7D, removed from the second mould.

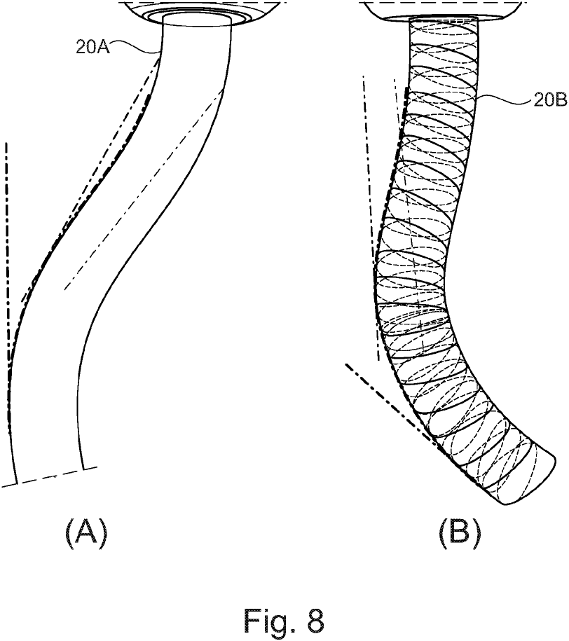

FIG. 8A shows a CM 20A without helical reinforcement. When subjected to a magnetic field, the unreinforced CM has a mean twist of 145◦ ± 12◦ (where 180◦ would indicate a complete reversal of the permanent magnets) and mean bend is just 6◦ ± 5◦. FIG. 8B shows a CM 20B with helical reinforcement. In the reinforced CM 20B mean twist is reduced to 49◦ ±5 ◦ and, due to preservation of magnetic energy, mean bend increases to 40◦ ±7◦. In other words, when unreinforced 20A and reinforced 20B CMs are subjected to identical magnetic fields, the unreinforced CM 20A twists, without producing a sufficient bend angle, whereas the reinforced CM 20B twists significantly less, and produces the required bend angle.

The magnetic elements are magnetised, before clinical use, with a magnetic profile that can be actuated during clinical use in order to determine the shape of the CM. The CM may be designed to have a specific predetermined shape that can be “switched on” by the external magnetic field when the CM has reached its destination. Alternatively, the CM may be designed with a specific insertion profile that can be dynamically controlled by the external magnetic field and a magnetic field gradient so that each segment moves in a “follow my leader” fashion to avoid obstructions and to follow a desired path during insertion.

Independent control of the magnetic elements enables the CM to adopt a shape along its length that can be selected for the specific clinical application and indeed the anatomical structures of a specific patient. This enables the CM to adopt a shape conforming to tortuous curvilinear trajectories without exerting significant pressure on surrounding tissues. Control along the length of the CM provides the ability to stiffen part(s) of the CM to accomplish specific surgical tasks that need structural rigidity.

The magnetic elements can have homogenous magnetisation i.e. identical magnetisation for each element, tuneable magnetisation i.e. where the magnetisation can be changed dynamically, or heterogenous magnetisation i.e. where each element has a different magnetisation profile.

In order to actuate the shape-forming aspects of the CM, an external magnetic field and, optionally, a magnetic field gradient is applied. Magnetic fields offer the possibility of manipulating the CM from afar and with penetrate human tissues without inflicting any harm on the patient. Magnetic control of a CM avoids the need for tendons or other internal actuation mechanisms thus facilitating miniaturisation and body flexibility of the CM.

The external magnetic fields and magnetic field gradients can be either uniform in the entire workspace or position-variant. This gives the following example combinations:
Homogenous magnetisation, uniform fields and position-variant field gradients;
Homogenous magnetisation and position-variant fields generated by permanent magnets;
Homogenous magnetisation and position-variant fields generated by electromagnets;
Tuneable magnetisation and uniform fields;
Heterogeneous magnetisation and uniform fields;
Heterogeneous magnetisation, position-variant fields and position-variant field gradients.

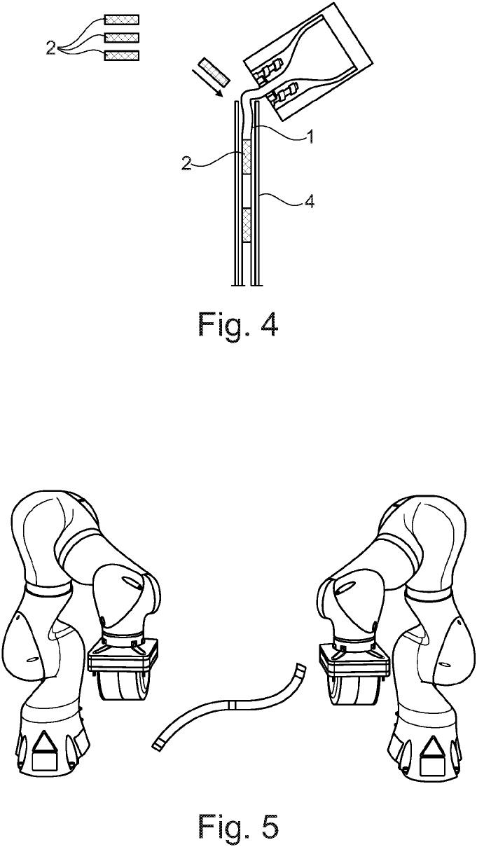

The external magnetic fields and magnetic field gradients can be provided by any one of a number of different techniques, for example: electromagnetic coils, MRI (magnetic resonance imaging) or multiple arm collaborative magnetic manipulation. Use of dual arm manipulation is schematically illustrated in FIG. 5 but more than two arms could be used.

Reducing the volume of the magnetic elements of the CM in order to facilitate miniaturisation leads to a loss of magnetic wrench for a given field. However this can be directly compensated for through appropriate dimensioning of the external magnetic actuation system. Specifically, more force/torque can be achieved by using more powerful actuation systems without a direct increase in the CM’s dimensions.

For completeness, the complete content of the priority document of the present application is reproduced below and forms part of the description of the present application. Claims follow thereafter.

1. Magnetic shape-forming surgical continuum manipulator (“CM”) comprising an elastomeric base material and a plurality of magnetic elements, the plurality of magnetic elements being located at a plurality of points along a length of the CM and each magnetic element having a predetermined magnetic profile, whereby the shape of the CM can be magnetically manipulated substantially along said length by the application of an external magnetic field and, optionally, a magnetic field gradient.

2. Magnetic shape-forming surgical continuum manipulator according to claim 1 wherein the plurality of magnetic elements comprises magnetic particles dispersed in the elastomeric base material.

3. Magnetic shape-forming surgical continuum manipulator according to claim 2 wherein the magnetic particles are dispersed at different concentrations and/or have different magnetic profiles along said length.

4. Magnetic shape-forming surgical continuum manipulator according to claim 1 wherein the plurality of magnetic elements comprises multiple spaced permanent magnets embedded in the elastomeric base material.

5. Magnetic shape-forming surgical continuum manipulator according to any of the preceding claims further comprising a lumen along said length providing a working channel therethrough.

6. Magnetic shape-forming surgical continuum manipulator according to any of the preceding claims having an external diameter of less than 2 mm.

7. Magnetic shape-forming surgical continuum manipulator according to any of the preceding claims further comprising one or more sensors.

8. Magnetic shape-forming surgical continuum manipulator according to any of the preceding claims wherein said elastomeric base material has an anisotropic elasticity distribution.

9. Magnetic shape-forming surgical continuum manipulator according to any of the preceding claims further comprising a reinforcing element having higher stiffness than said elastomeric based material.

10. Magnetic shape-forming surgical continuum manipulator according to claim 9 wherein said reinforcing element comprises a helical element.

11. Method of manufacturing a magnetic shape-forming surgical continuum manipulator according to any of the preceding claims comprising the steps of:
a. Combining said magnetic elements with the elastomeric material by dispersing or embedding said magnetic elements therein; and
b. Magnetizing said magnetic elements to create said predetermined magnetic profile.

12. Method according to claim 11 wherein said combining step comprises extruding said elastomeric material.

13. Method according to claim 11 wherein said combining step comprises moulding said elastomeric material in a shaped tray.

14. Method according to any of claims 11-14 wherein said combining step is performed before said magnetizing step.

15. Method according to any of claims 11-14 wherein said combining step is performed after said magnetizing step.

16. Method according to any of claims 11-15 further comprising the application of a helical reinforcing element to the elastomeric base material in order to restrict torsion whilst allowing bending thereof.

17. Method of controlling a magnetic shape-forming surgical continuum manipulator according to any of claims 1-10 comprising the steps of:
a. applying an external magnetic field to the CM;
b. allowing the CM to adopt a shape along the length thereof as a result of manipulating said external magnetic field.

18. Method as claimed in claim 17 further comprising the step of pulling the CM to a new location as a result of the application and/or manipulation of said external magnetic field.

19. Method as claimed in claim 18 wherein a pulling force is applied along the length of the CM.

20. Method as claimed in any of claims 17-19 wherein, in step b, the CM adopts a stiffened shape in order to provide a working channel via said lumen.

21. Method as claimed in any of claims 17-20 wherein, in step b, the CM adopts a dynamically changing shape dependent on said manipulation of the external magnetic field.

22. Method as claimed in any of claims 17-21 wherein said external magnetic field is applied by dual arm collaborative magnetic manipulation.

23. Method as claimed in any of claims 17-21 wherein said external magnetic field is applied by electromagnetic coils.

24. Method as claimed in any of claims 17-21 wherein said external magnetic field is applied by magnetic resonance imaging (MRI).
